(19) United States
(12) Patent Application Publication
(10) Pub. No.: US 2006/0258750 A1
(43) Pub. Date: Nov. 16, 2006

(54) Use of bicyclo[2.2.1]heptane derivatives for the preparation of neuroprotective pharmaceutical compositions

(76) Inventors: Istvan Gacsalyi, Budapest (HU); Gabor Gigler, Budapest (HU); Laszio Gabor Harsing, Budapest (HU); Gyorgy Levay, Budakeszi (HU); Krisztina Moricz, Budapest (HU); Annamaria Simo, Erd (HU); Gabor Szenasi, Budapest (HU)

Correspondence Address: THE FIRM OF KARL F ROSS, 5676 RIVERDALE AVENUE, PO BOX 900, RIVERDALE (BRONX), NY, 10471-0900, US

(21) Appl. No.: 10/562,393

(22) PCT Filed: Jun. 22, 2004
(86) PCT No.: PCT/HU04/00062

§ 371 Date: Apr. 25, 2006

(30) Foreign Application Priority Data
Jun. 23, 2003 (HU) P 0301906

(51) Int. Cl. A61K 31/138

(52) U.S. Cl. 514/650

(57) ABSTRACT

The invention relates to the use of compounds of the general Formula (I), wherein R³ stands for hydrogen or hydroxy; R¹ stands for hydrogen or alkyl; and R² stands for alkyl) and pharmaceutically acceptable acid addition salts for the preparation of pharmaceutical compositions having neuroprotective effect.

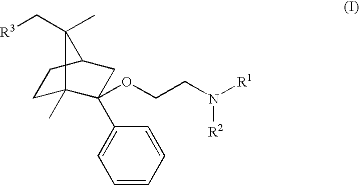

### TECHNICAL BACKGROUND OF THE INVENTION

The present invention relates to a new therapeutical use of bicyclo[2.2.1]heptane derivatives. More particularly the present invention is concerned with the use of bicyclo[2.2.1]heptane derivatives for the preparation of pharmaceutical compositions having neuroprotective effect.

### STATE OF THE ART

It is known that 1,1,7-trimethyl-dicyclo[2.2.1]heptane derivatives comprising a phenyl, phenyl alkyl or thienyl side-chain in position 2 possess anticonvulsive, motility inhibiting, hexobarbital narcosis potentiating and analgetic effect (GB 2,065,122). An outstanding member of said compound group (1R,2S,4R)-(−)-2-(2-dimethylaminoethoxy)-2-phenyl-1,7,7-trimethyl-bicyclo[2.2.1]heptane in the form of the free base and pharmaceutically acceptable salts thereof—particularly the fumarate—was disclosed in HU 212,547.

Deramciclane showed considerable effects in different animal models of anxiety and stress. In the Vogel punished drinking test deramciclane was active in 1 and 10 mg/kg after oral administration [Gacsályi et. al, Receptor binding profile and anxiolytic activity of deramciclane (EGIS-3886) in animal models, Drug Dev. Res. 40: p. 338-348, (1997)]. In the social interaction model, the compound increased the time spent with social interactions after a single 0.7 mg/kg oral treatment. In the light-dark test, [Crawley, J. N. Neuropharmacological specifity of a simple model of anxiety for the behavioural actions of benzodiazepine, Pharmacol. Biochem. Behavior, 15: p. 695-699 (1981)] deramciclane proved to be active at a single oral dose of 3 mg/kg sc. In the marble burying test [Broekkamp, C. L. et al, Major Tranquillizers Can Be Distinguished from Minor Tranquillisers on the Basis of Effects on Marble Burying and Swim-Induced Grooming in Mice. Eur. J. Pharmacol. 126: p. 223-229, (1986)]the molecule was active in 10 and 30 mg/kg after oral treatment.

Deramciclane was ineffective in the elevated plus maze test, but it antagonized anxiety caused by CCK in this test [Gacsályi et. al, Receptor binding profile and anxiolytic activity of deramciclane (EGIS-3886) in animal models, Drug Dev. Res. 40: p. 338-348, (1997)].

Besides these anxiolytic effects, deramciclane produced antidepressant activity at 1 and 10 mg/kg ip. doses in the learned helplessness test, which is a known animal model of depression [Grial et al., Biol. Psychiatry, 23, 237-242 (1988)].

Based on its receptor profile, deramciclane binds primarily to central 5-HT_{2C} and 5-HT_{2A} receptors. Anxiolytic and antidepressant effects of deramciclane can be explained by its affinity for these 5-HT receptors.

High purity deramciclane of the Formula 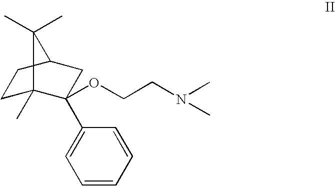
comprising less than 0.2% of (1R,3S,4R)-3-[2-(N,N-dimethylaminoethyl)]-1,7,7-trimethyl-bicyclo[2.2.1]heptane-2-one of the Formula 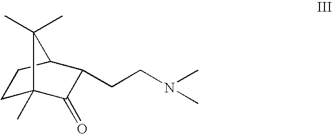
is described in EP 1,052,245.

The N-methyl derivative of deramciclane of the Formula II is described in WO 98/17230. This compound exhibits valuable anxiolytic effect.

According to an aspect of the present invention there is provided the use of bicyclo[2.2.1.]heptane derivatives of the general Formula 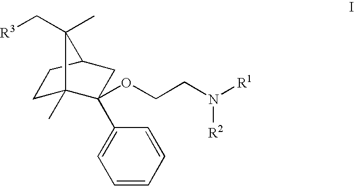
(wherein
R³ stands for hydrogen or hydroxy;
R¹ stands for hydrogen or alkyl; and
R² stands for alkyl)
   and pharmaceutically acceptable acid addition salts for the preparation of pharmaceutical compositions having neuroprotective effect.

The present invention is based on the recognition that compounds of general Formula I produce protection against neuronal injury induced by global cerebral ischemia and consequential pathological changes in behavioural parameters (spontaneous motility). This effect is independent of its known mode of action and of its anxiolytic and stress-reducing effects since ritanserin, a 5-HT_{2A/2C} antagonist, i.e. a compound with comparable mode of action to deramciclane, did not show neuroprotective activity in a similar ischemia model (Piera, M. J., et. al, Lack of efficacy of 5-HT2A receptor antagonists to reduce brain damage after 3 minutes of transient global cerebral ischaemia in gerbils, Fundam. Clin. Pharmacol, 9: p. 562-568, 1995). This effect of compounds of general Formula I make them suitable for the treatment of conditions in consequence of acute brain and spinal damages e.g. stroke, cerebral vasospasm, and of neuronal death succeeding head and spinal injuries caused by accidents as well as make them suitable for the improvement of behavioural parameters induced by neuronal loss and also for the treatment of chronic neurodegenerative disorders e.g. multiple sclerosis, motoneuron disease (amyoptrophic lateral sclerosis, ALS), Creutzfeld-Jakob diseases etc.

The definition of the terms used in the present patent specification is the following unless otherwise specified.

The term “lower alkyl” relates to straight or branched chain saturated aliphatic hydrocarbon group containing 1-4 carbon atom, e.g. methyl, ethyl, n-propyl, isopropyl, n-butyl, secondary butyl etc.

The term “pharmaceutically acceptable acid addition salts” relates to salts formed with pharmaceutically acceptable non-toxical inorganic or organic acids. For salt formation e.g. hydrochloric acid, hydrogen bromide, sulfuric acid, phosphoric acid, acetic acid, formic acid, lactic acid, tartaric acid, maleic acid, malic acid, amygdalic acid, fumaric acid, benzenesulfonic acid, p-toluene sulfonic acid etc. can be used. Salts formed with fumaric acid are particularly preferable.

A preferable embodiment of our invention is the utilization of compounds of general Formula I and their acid addition salts for the preparation of pharmaceutical compositions suitable for the reduction of the consequences of acute ischemic or traumatic brain and spinal damage, especially the various types of stroke or cerebral vasospasm, severe brain vessel occlusion, neuronal loss and its functional consequences in the case of head and spinal injuries caused by accidents.

A further preferable embodiment of our invention is the utilization of compounds of general Formula I or of their acid addition salts for the preparation of pharmaceutical compositions suitable for the treatment of neurodegenerative disorders.

A further preferable embodiment our invention is the utilization of compounds of general Formula I or acid addition salts thereof for the preparation of pharmaceutical compositions suitable for the treatment of motoneuron disease (ALS), sclerosis multiplex or Creutzfeld-Jakob disease.

A further preferable embodiment of our invention is the utilization of compounds of general Formula I or their acid addition salts for the preparation of pharmaceutical compositions suitable for the prevention of stroke; preventive treatment can be started after the event of first stroke.

The neuroprotective dose of the compounds of the general Formula I can be varied between broad ranges and depends on various factors e.g. the activity of the given active ingredient, the body weight, age and condition of the patient to be treated, the seriousness of the treated disease, the form of administration is always determined by the physician. The daily neuroprotective dose is preferably between about 0.1 mg/kg and 150 mg/kg, particularly between about 1 mg/kg and about 150 mg/kg, particularly advantageously between about 10 mg/kg and about 150 mg/kg.

As compounds of the general Formula I preferably (1R,2S,4R)-(−)-2-(2-dimethylaminoethoxy)-2-phenyl-1,7,7-trimethyl-bicyclo[2.2.1]heptane or pharmaceutically acceptable acid addition salts thereof, particularly (1R,2S,4R)-(−)-2-(2-dimethylaminoethoxy)-2-phenyl-1,7,7-trimethyl-bicyclo[2.2.1]heptane-fumarate can be used.

Further compounds of the general Formula I which can be preferably used in accordance with the present invention are the following:
(1R,2S,4R)-(−)-2-(2-methylaminoethoxy)-2-phenyl-1,7,7-trimethyl-bicyclo[2.2.1]heptane;
(1R,2S,7R)-2-phenyl-2-(2-methylaminoethoxy)-7-hydroxymethyl-1,7-dimethyl-bicyclo[2.2.1]heptane; or
(1R,2S,7R)-2-phenyl-2-(2-ethylaminoethoxy)-7-hydroxymethyl-1,7-dimethyl-bicyclo[2.2.1]heptane
   or pharmaceutically acceptable acid addition salts of the above compounds.

According to the most preferred embodiment of the present invention (1R,2S,4R)-(−)-2-(2-dimethylaminoethoxy)-2-phenyl-1,7,7-trimethyl-bicyclo[2.2.1]heptane of the Formula II or the pharmaceutically acceptable acid addition salts thereof, particularly (1R,2S,4R)-(−)-2-(2-dimethylaminoethoxy)-2-phenyl-1,7,7-trimethyl-bicyclo[2.2.1]heptane-fumarate can be used for the preparation of neuroprotective pharmaceutical compositions.

According to a particularly preferred embodiment of the present invention as compound of the general Formula I (1R,2S,4R)-(−)-2-(2-dimethylaminoethoxy)-2-phenyl-1,7,7-trimethyl-bicyclo[2.2.1]heptane of the Formula II or a pharmaceutically acceptable acid addition salt thereof containing not more than 0.2% of (1R,3S,4R)-3-[2-(N,N-dimethylaminoethyl)]-1,7,7-trimethyl-bicyclo[2.2.1]heptane-2-one of the Formula III or a pharmaceutically acceptable acid addition salt thereof is used.

According to a very preferable variant of the above embodiment of the present invention as compound of the general Formula I (1R,2S,4R)-(−)-2-(2-dimethylaminoethoxy)-2-phenyl-1,7,7-trimethyl-bicyclo[2.2.1]heptane-fumarate containing not more than 0.2% of (1R,3S,4R)-3-[2-(N,N-dimethylaminoethyl)]-1,7,7-triethyl-bicyclo[2.2.1]heptane-2-one-fumarate is used.

According to a further aspect of the present invention there are provided neuroprotective pharmaceutical compositions comprising as active ingredient a compound of the general Formula I (wherein R¹, R² and R³ are as stated above) or a pharmaceutically acceptable acid addition salt thereof in admixture with pharmaceutically acceptable solid or liquid pharmaceutical carriers and/or auxiliary agents.

The pharmaceutical compositions of the present invention can be prepared by known methods of the pharmaceutical industry. Thus one may proceed by admixing a compound of the general Formula I or a pharmaceutically acceptable acid addition salt thereof with inert solid or liquid pharmaceutical carriers and/or auxiliary agents and bringing the mixture into galenic form.

The neuroprotective pharmaceutical compositions according to the present invention can be administered orally (tablets, coated tablets, hard or soft gelatine capsules, solutions, suspensions etc.), parenterally (e.g. subcutaneous, intramuscular, intravenous injections), rectally (e.g. suppositories) or nasally (e.g. aerosols). The active ingredient can be delivered promptly from the pharmaceutical compositions in which case the duration of therapeutical effect is practically determined by the duration of the active ingredient per se. However, the neuroprotective pharmaceutical compositions of the present invention can also be prepared in sustained release form, wherein the duration of the therapeutical effect is affected by the form of the composition too (pharmaceutical compositions of regulated, sustained or delayed active ingredient delivery).

The pharmaceutical compositions of the present invention can be prepared by conventional methods of pharmaceutical industry.

The tablets and capsules can contain lactose (monohydrate, anhydrate, powdered, dried etc.) mannitol, cellulose type (powdered, microcrystalline etc.) as filler. Gelatine, polyvinyl pyrrolidone (having different molecular weight), cellulose ether type (hydroxypropyl cellulose, hydroxypropyl methylcellulose, methyl cellulose, ethyl cellulose etc.), hydrolyzed starch, vegetable gum (gum arabic, guar gum etc.) can be used in aqueous solution or in solution formed with aliphatic alcohols having 1-4 carbon atoms in mixture of said solvents as binder. The disintegrant used can be starch (potato starch, maize starch, wheat starch etc.) or a so-called super disintegrant, e.g. carboxymethyl cellulose (commercial name Ac-di-sol), sodium carboxymethyl starch (commercial name Primojel, Ultraamilopektin, Explo-Tab), polyvinyl pyrrolidone (commercial name Poliplasdone) etc. As lubricant e.g. alkali stearates (such as magnesium stearate, calcium stearate), fatty acids (e.g. stearic acids), glycerides (commercial name Precirol, Cutina H), paraffin oil, silicon oil, silicon oil emergents (talc, silica etc.) can be used. The active ingredients and auxiliary agents can be prepared for use in the compressing and anticapsulating procedure by liquid or dry granulating process or filtered powder homogenization.

Regulated or sustained release solid pharmaceutical compositions can be prepared by known methods of pharmaceutical industry. Such compositions may be tablets containing various retardizing components [e.g. hydrophilic polymers, such as hydroxypropyl cellulose, hydroxypropyl methylcellulose, carboxymethyl cellulose, polyacrylic derivatives, polysaccharoses (e.g. guar gum, xanthan gum) etc. and mixtures thereof] or hydrophobic polymers (e.g. ethyl cellulose, methacrylic ester copolymers, polyvinyl acetate, polyvinyl butyral etc.) and mixtures thereof. In other neuroprotective pharmaceutical compositions of the present invention the retardizing effect is achieved by using a matrix which comprises a mixture of hydrophilic and hydrophobic polymers, or a mixture of polymers and fatty substances. The tablets can also be prepared in multilayer forms wherein the active ingredients are incorporated into separate layers and thus the dissolution profile of the active ingredient can be better adjusted to the specific pharmacokinetical characteristics thereof.

The sustained release neuroprotective pharmaceutical compositions of the present invention can also be prepared in the form of coated pellets. The preparation of the pellets can be performed separately from the active ingredient or from a mixture of the active ingredient. The preparation of the pellets can be performed by extrusion or by spheronification rotogranulating methods or by coating the layers on placebo pellets. A coating of the pellets can be carried out in rotating fluidizing equipment. As coating agent solutions or dispersents of water insoluble polymers formed with organic solvents (preferably aliphatic alcohols containing 1-3 carbon atoms and/or chlorinated hydrocarbons containing 1-2 carbon atoms and/or acetone and/or ethylacetate or mixtures thereof) can be used.

The neuroprotective pharmaceutical compositions according to the present invention can also be prepared and used in the form of osmotic or diffusion-osmotic compositions. Tablets containing the active ingredient and hydrophilic polymers (e.g. hydroxypropyl methyl cellulose) are prepared which are coated with a film-layer either semipermeable (e.g. cellulose acetate) or permeable (e.g. amino methacrylate copolymer) for the active ingredient, thereafter an orifice is formed in said layer, through which the active ingredient is optically pushed out into the aqueous medium.

According to a further aspect of the present invention there is provided the use of the compounds of the general Formula and pharmaceutically acceptable acid addition salts thereof as neuroprotective pharmaceutical active ingredients.

The compounds of the general Formula and pharmaceutically acceptable acid addition salts thereof can be used particularly for the reduction of the consequences of acute ischemic or traumatic brain and spinal damage, especially in the various types of stroke or cerebral vasospasm, severe brain vessel occlusion, neuronal loss and its functional consequences in the case of head and spinal injuries caused by accidents; or for the treatment of neurodegenerative disorders; or for the treatment of motoneuron disease (ALS), sclerosis multiplex or Creutzfeld-Jakob disease; or for the prevention of stroke; whereby preventive treatment can be started after the event of first stroke.

According to a further aspect of the present invention there is provided a neuroprotective method of treatment which comprises administering to the patient in need of such treatment a pharmaceutically acceptable amount of a compound of the general Formula I or a pharmaceutically acceptable salt thereof, preferably (1R,2S,4R)-(−)-2-(2-dimethylaminoethoxy)-2-phenyl-1,7,7-trimethyl-bicyclo[2.2.1]heptane of the Formula II or a pharmaceutically acceptable acid addition salt thereof.

The neuroprotective effect of the compounds of the general Formula I is shown by the following tests. As compound of the general Formula (1R,2S,4R)-(−)-2-(2-dimethylaminoethoxy)-2-phenyl-1,7,7-trimethyl-bicyclo[2.2.1]heptane-fumarate (deramciclane fumarate) is used.

The neuroprotective effect of deramciclane was demonstrated in a model of global cerebral ischemia induced by bilateral carotid occlusion. In our experiments male Mongolian gerbils weighing 50-80 g were used. Deramciclane was administered at 3×30 mg/kg intraperitoneally 60 min before, 30 and 90 min after surgery. Deramciclane was suspended in 0.4% methyl-cellulose solution. In ether narcosis, the right and left common carotid arteries were exposed through an anterior midline cervical incision and isolated from the vagus nerves and the surrounding tissues. Full arrest of carotid blood flow was achieved by tightening an aneurysm clip for 3 min. During surgery the body temperature of the animals was kept at the individual preoperative level (37.5±0.5° C.) with the help of a heating pad and a heating lamp.

Since it is well known that global cerebral ischemia induces hyperactivity in animals, which was found to be closely correlated with the severity of hippocampal damage (Gerhardt, S. C. et. al, Motor activity changes following cerebral ischemia in gerbils are correlated with the degree of neuronal degeneration in hippocampus, Behav. Neurosci. 102: p. 301-303, 1988), four days after surgery the locomotor activity of the animals were measured in a symmetrical Y-maze (arms were 40 cm long and 10 cm wide with 21.5 cm high walls). The Mongolian gerbils were placed in the centre of the maze then the number of entries into the three arms was recorded for 5 min. By definition, the animal performed an arm entry when it entered the arm and proceeded at least the distance of its body length. The gerbil was considered to exit the arm when it left it fully. Differences between groups were statistically evaluated by Kruskal-Wallis ANOVA. In case of p<0.05 significance Mann-Whitney U-test was used for paired comparisons.

After behavioural testing the animals were anesthetized with 60 mg/kg i.p. pentobarbital (10 ml/kg) and perfused through the heart first with saline then with a fixative solution containing 0.1% glutaraldehyde, 4% paraformaldehyde, and 0.2% picric acid in 0.1 M phosphate buffer pH 7.4 for 30 min. The brain was removed from the skull and post-fixed for at least 1 week at 4° C. in the same fixative solution.

Alternate coronal sections of 60 μm thickness were cut from different levels of the dorsal hippocampus by a microtone. The sections were repeatedly washed in 0.1 M phosphate buffer then stained by silver impregnation.

The sections were examined under light microscopy and the overall neuronal damage in the hippocampal CA1 subfield in both hippocampi was scored on a 6-point scale: (0) undamaged, (1)<10%, (2) 10-30%, (3) 30-50%, (4) 50-70%, (5) 70-90%, and (6) 90-100% cell loss. Group differences between drug-treated and vehicle-treated groups were statistically analysed by Mann-Whitney U-test. Our results are summarized in Table 1.

TABLE 1
Effect of deramciclane on hippocampal CA1 pyramidal cell
death and hypermotility of animals following 3 min bilateral
carotid artery occlusion (BCO) induced global ischemia

| | Dose | CA1 cell | | Number | |
|---|---|---|---|---|---|
| | mg/kg | death | | of arm | |
| Treatment | i.p. | (score) | Effect % | entries | Effect % |
| Sham | — | — | — | 40.25 | — |
| BCO | — | 4.90 | — | 65.44⁺⁺ | — |
| BCO + | 3 × 30 | 0.89** | −82% | 38.78** | −100 |
| deramciclane | | | | | |

⁺⁺p < 0.01 statistical significance, compared to the sham-operated group (Mann-Whitney U-test, following Kruskal-Wallis ANOVA),
**p < 0.01 statistical significance, compared to the BCO group (Mann-Whitney U-test, following Kruskal-Wallis ANOVA).

The above results proved that deramciclane in the applied dose significantly reduced the proportion of cell death in the hippocampal CA1 region and decreased the locomotor activity of animals into the normal range in parallel to the improvement of histological score. Deramciclane was not only protective against neuronal cell death but it was also effective in normalizing the clinically important behavioural anomalies.

On the basis of our observations in animal experiments, deramciclane protected against neuronal loss induced by global cerebral ischemia as well as against behavioural anomalies developed in consequence of neuronal death. This surprising effect of deramciclane was not possible to be predicted because ritanserin, which also had a 5-HT_{2A/2C} mode of action and anxiolitic effect in animal experiments, did not produce neuroprotective effect in this model.

In summary, according to the recognition described in the present invention deramciclane possessed neuroprotective activity because the compound considerably reduced neuronal cell death in the CA1 region of the hippocampus as well as reduced hyperactivity, which was the consequence of neuronal death observed four days after global cerebral ischemia caused by bilateral common carotid artery occlusion in Mongolian gerbils. Based on all the aforementioned, the therapeutic application of deramciclane can be favourable for the treatment of acute ischemic or traumatic brain and spinal cord damages e.g. different forms of stroke, cerebral vasospasm, severe cerebral vessel stenosis, accident-related head and spinal cord damages etc., that can reduce the extent of neuronal destruction thereby the gravity of functional deficit caused by neuronal loss, furthermore, for the treatment of chronic neurodegenerative diseases e.g. amyotrophic lateral sclerosis (ALS), multiple sclerosis and Creutzfeld-Jakob disease, etc., that is for the deceleration or stopping the rate of neuronal death, thereby the progression of diseases in all disease states or statuses, in which some or all neurons or the part of them are damaged or killed.

Further details of the present invention are to be found in the following Examples without limiting the scope of protection to said Examples.

### EXAMPLE 1

Tablets having the following composition are prepared by known methods of pharmaceutical industry:

| Component | Amount, mg/tablet |
|---|---|
| Deramciclane | 20 |
| Maize starch | 90 |
| Polyvinyl pyrrolidone | 68 |
| Magnesium stearate | 2 |
| Total weight | 180 |

### EXAMPLE 2

Gelatine capsules having the following composition are prepared by known methods of pharmaceutical industry:

| Component | Amount, mg/capsule |
|---|---|
| Deramciclane | 20 |
| Maize starch | 212 |
| Aerosil ® | 5 |
| Magnesium stearate | 3 |
| Total weight | 240 |

tightening an aneurysm clip for 3 min. During surgery the body temperature of the animals was kept at the individual preoperative level (37.5±0.5° C.) with the help of a heating pad and a heating lamp.

Since it is well known that global cerebral ischemia induces hyperactivity in animals, which was found to be closely correlated with the severity of hippocampal damage (Gerhardt, S. C. et. al, Motor activity changes following cerebral ischemia in gerbils are correlated with the degree of neuronal degeneration in hippocampus, Behav. Neurosci. 102: p. 301-303, 1988), four days after surgery the locomotor activity of the animals were measured in a symmetrical Y-maze (arms were 40 cm long and 10 cm wide with 21.5 cm high walls). The Mongolian gerbils were placed in the centre of the maze then the number of entries into the three arms was recorded for 5 min. By definition, the animal performed an arm entry when it entered the arm and proceeded at least the distance of its body length. The gerbil was considered to exit the arm when it left it fully. Differences between groups were statistically evaluated by Kruskal-Wallis ANOVA. In case of p<0.05 significance Mann-Whitney U-test was used for paired comparisons.

After behavioural testing the animals were anesthetized with 60 mg/kg i.p. pentobarbital (10 ml/kg) and perfused through the

What we claim is:

1. Use of compounds of the general Formula 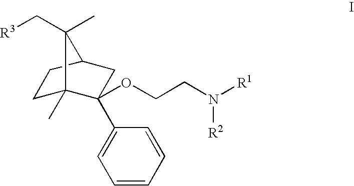
(wherein
R³ stands for hydrogen or hydroxy;
R¹ stands for hydrogen or alkyl; and
R² stands for alkyl)
and pharmaceutically acceptable acid addition salts for the preparation of pharmaceutical compositions having neuroprotective effect.

2. Use according to claim 1 for the preparation of pharmaceutical compositions suitable for the reduction of the consequences of acute ischemic or traumatic brain and spinal damages, especially the various types of stroke or cerebral vasospasm, severe brain vessel occlusion, neuronal loss and its functional consequences in the case of head and spinal injuries caused by accidents.

3. Use according to claim 1 for the preparation of pharmaceutical compositions having chronical neurodegenerative effect.

4. Use according to claim 3 for the preparation of pharmaceutical composition suitable for the treatment of motoneuron disease (ALS), sclerosis multiplex or Creutzfeld-Jakob disease.

5. Use according to any of claims 1-4 wherein (1R,2S,4R)-(−)-2-(2-dimethylaminoethoxy)-2-phenyl-1,7,7-trimethyl-bicyclo[2.2.1]heptane (deramciclane) or a pharmaceutically acceptable acid addition salt is used as compound of the general Formula I.

6. Use according to claim 5 wherein (1R,2S,4R)-(−)-2-(2-dimethylaminoethoxy)-2-phenyl-1,7,7-trimethyl-bicyclo[2.2.1]heptane-fumarate (deramciclane-fumarate) is used as compound of the general Formula I.

7. Use according to claim 1 wherein (1R,2S,4R)-(−)-2-(2-dimethylaminoethoxy)-2-phenyl-1,7,7-trimethyl-bicyclo[2.2.1]heptane of the Formula 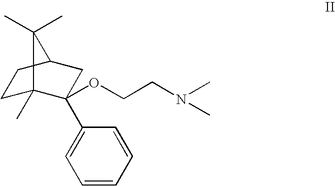
or a pharmaceutically acceptable acid addition salt containing not more than 0.2% of (1R,3S,4R)-3-[2-(N,N-dimethylaminoethyl)]-1,7,7-trimethyl-bicyclo[2.2.1]heptane-2-one of the Formula 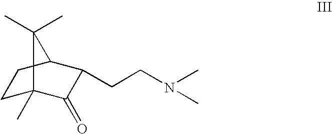
or a pharmaceutically acceptable acid addition salt thereof is used as compound of the general Formula I.

8. Use according to claim 7 wherein (1R,2S,4R)-(−)-2-(2-dimethylaminoethoxy)-2-phenyl-1,7,7-trimethyl-bicyclo[2.2.1]heptane-fumarate containing not more than 0.2% of (1R,3S,4R)-3-[2-(N,N-dimethylaminoethyl)]-1,7,7-trimethyl-bicyclo[2.2.1]heptane-2-one-fumarate is used as compound of the general Formula I.

9. Use according to any of claims 1-4 wherein
(1R,2S,4R)-(−)-2-(2-methylaminoethoxy)-2-phenyl-1,7,7-trimethyl-bicyclo[2.2.1]heptane;
(1R,2S,7R)-2-phenyl-2-(2-methylaminoethoxy)-7-hydroxymethyl-1,7-dimethyl-bicyclo[2.2.1]heptane; or
(1R,2S,7R)-2-phenyl-2-(2-ethylaminoethoxy)-7-hydroxymethyl-1,7-dimethyl-bicyclo[2.2.1]heptane
or a pharmaceutically acceptable acid addition salt thereof is used as compound of the general Formula I.

10. Neuroprotective pharmaceutical composition comprising as active ingredient a compound of the general Formula I (wherein R¹, R² and R³ are as stated in claim 1) or a pharmaceutically acceptable acid addition salt thereof in admixture with inert pharmaceutically acceptable solid or liquid pharmaceutical active ingredient and/or auxiliary agent.

11. Pharmaceutical composition according to claim 10 suitable for the reduction of the consequences of acute ischemic or traumatic brain and spinal damage, especially the various types of stroke or cerebral vasospasm, severe brain vessel occlusion, neuronal loss and its functional consequences in the case of head and spinal injuries caused by accidents.

12. Pharmaceutical composition according to claim 10 suitable for the treatment of neurodegenerative diseases.

13. Pharmaceutical composition according to claim 11 suitable for the treatment of motoneuron disease (ALS), sclerosis multiplex or Creutzfeld-Jakob disease.

14. Pharmaceutical composition according to any of claim 10-13 comprising (1R,2S,4R)-(−)-2-(2-dimethylaminoethoxy)-2-phenyl-1,7,7-trimethyl-bicyclo[2.2.1]heptane of the Formula II or a pharmaceutically acceptable acid addition salt as compound of the general Formula I.

15. Pharmaceutical composition according to claim 14 comprising (1R,2S,4R)-(−)-2-(2-dimethylaminoethoxy)-2-phenyl-1,7,7-trimethyl-bicyclo[2.2.1]heptane-fumarate as compound of the general Formula I.

16. Use of compounds of the general Formula I (wherein R¹, R² and R³ are as stated in claim 1) and pharmaceutically acceptable acid addition salts thereof as neuroprotective pharmaceutical active ingredient.

17. Use according to claim 16 for the reduction of the consequences of acute ischemic or traumatic brain and spinal damages, especially the various types of stroke or cerebral vasospasm, severe brain vessel occlusion, neuronal loss and its functional consequences in the case of head and spinal injuries caused by accidents.

18. Use according to claim 16 for the treatment of chronical neurodegenerative diseases.

19. Use according to claim 16 for the treatment of motoneuron disease (ALS), sclerosis multiplex or Creutzfeld-Jakob disease.

20. Use of (1R,2S,4R)-(−)-2-(2-dimethylaminoethoxy)-2-phenyl-1,7,7-trimethyl-bicyclo[2.2.1]heptane of the Formula II and pharmaceutically acceptable acid addition salts thereof in the indications according to claims 16-19.

21. Use of (1R,2S,4R)-(−)-2-(2-dimethylaminoethoxy)-2-phenyl-1,7,7-trimethyl-bicyclo[2.2.1]heptane-fumarate in the indications according to claim 16-19.

22. Neuroprotective method of treatment which comprises administering to the patient in need of such treatment a compound of the general Formula I or a pharmaceutically acceptable acid addition salt thereof, perferably (1R,2S,4R)-(−)-2-(2-dimethylaminoethoxy)-2-phenyl-1,7,7-trimethyl-bicyclo[2.2.1]heptane of the Formula II or a pharmaceutically acceptable acid addition salt thereof in a therapeuticly active amount.
